(19) United States
(12) Patent Application Publication
(10) Pub. No.: US 2007/0088321 A1
(43) Pub. Date: Apr. 19, 2007

(54) Use of bioabsorbable, biocompatible adhesives as a method of female contraception

(75) Inventor: James Quinn, Portola Valley, CA (US)

Correspondence Address: LEACH PATENT SERVICES, 5 EAST ALDINE DRIVE, HOCKESSIN, DE, 19707, US

(73) Assignee: Clast Trading LTD, Hong Kong (HK)

(21) Appl. No.: 11/240,932

(22) Filed: Sep. 30, 2005

(51) Int. Cl. A61M 31/00

(52) U.S. Cl. 604/515; 604/187

(57) ABSTRACT

Disclosed is a method of female contraception comprising: injecting a bioabsorbable and biocompatible tissue adhesive into the fallopian tubes, and allowing the said bioabsorbable tissue adhesive to polymerize to occlude the fallopian tubes (bilaterally) to achieve contraception. When the bioabsorbable biocompatible adhesive is one which forms a simple mechanical barrier, the contraception is temporary, and would be reversed upon bioabsorbtion of the tissue adhesive. When the bioabsorbable, biocompatible adhesive is one which produces a mild inflammatory reaction resulting in the formation of a permanent scar tissue plug, the contraception would be permanent.

### DESCRIPTION

In one embodiment of the invention, non-surgical sterilization of females may be achieved by introducing a bioabsorbable tissue adhesive into the fallopian tubes of the patient to occlude the fallopian tubes and thereby prevent conception. The bioabsorbable tissue adhesives may include mixtures of polyethylene glycol macromers or fibrin-based sealants. As used herein throughout this application, the term bioabsorbable tissue adhesive refers generically to tissue adhesives which are bioabsorbable and biocompatible.

Fallopian tubes have a complex anatomy and physiology. They are paired, hollow, seromuscular organs that extend from the superior-lateral aspect of the uterine fundus to the ovaries. The mucosa is thrown into folds that project into the lumen. The height and complexity of the folds vary among the segments of the tube. The mucosa is composed of an epithelia layer which is made up predominately of ciliated and secretory cells. Proportion of cell types vary within different regions of the oviduct. Ciliated cells predominate in the distal oviduct and decrease toward the uterus, whereas the inverse is true of secretory cells. Ciliary beat is toward the utereus. This shows the importance of the formulation ability to flow and fill when selecting the formulation for fallopian occlusion.

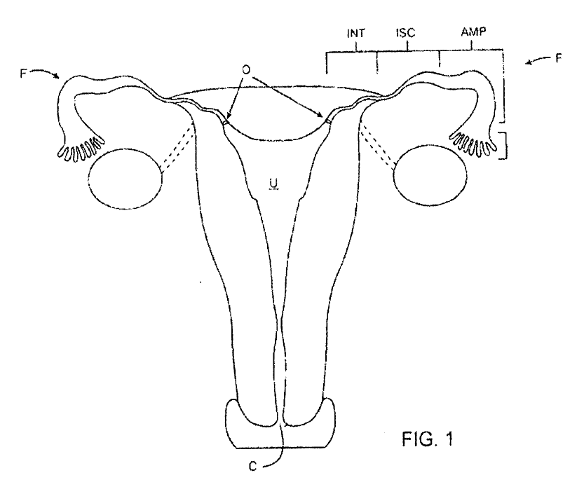

The length of the fallopian tube varies considerably from patient to patient, though in the human female, a minimum length of 8 cm and a maximum length of 14 cm are generally recognized as the upper and lower lengths, with 11 cm being average. The fallopian tube is divided into four sections as illustrated in FIG. 1: the intramural (INT), the isthmus (ISC), the ampulla (AMP) and the infundibulum (F). The intramural varies in length from 0.8 cm to 2 cm, and has a lumen cross-section which can vary from 0.2 mm to 2 mm. The isthmus cross section can vary from 2 mm to 3 mm at the ampulla junction to 0.2 mm to 2 mm at the intramural junction. The length of the isthmus can vary from 2 cm to 3 cm. The ampulla is the longest portion of the fallopian tube, varying in length from 5 cm to 8 cm. The lumen cross section in the this segment will vary from 1 mm to 10 mm. The infundibulum has a short trumpet shape with a maximum cross section of between 5 mm and 20 mm at its distal end, and can vary in length from 1 cm to 1.5 cm.

The fallopian tube is a well vascularized organ whose primary function is to transport ovum and spermatozoa in opposite directions and then transport the developing embryo into the uterus. In the infundibulum and ampulla, with their thin wall, large lumen, and complex densely ciliated mucosal folds, ciliary activity is of major importance in ovum transport, with contractual activity a subordinate effector. In contrast, transport through the isthmus with narrow lumen and sparsely ciliated endosalpinx appears to result primarily from contractile activity.

The ostium, (O) in FIG. 1, is the entrance from the Uterine cavity to the fallopian tubes intramural portion and is funnel shaped which aid access.

The configuration of the intramural portion is important to the present invention as in some embodiments of the invention, a catheter/guide wire is placed into this portion of the fallopian tube to allow delivery of the occluding adhesive.

Adhesives comprising mixtures of polyethylene glycol macromers are described in U.S. Pat. Nos. 6,624,245 and 6,312,725, the disclosures of which are herein incorporated by reference. Adhesives comprising mixtures of polyethylene glycol macromers are available commercially under the trade name CoSeal, from Cohesion Technologies, Palo Alto, Calif.

Fibrin-based sealants are comprised of a mixture of concentrated fibrinogen and thrombin. Fibrinogen molecules are composed of two sets of polypeptide chains, (A_{α}, B_{β, γ})₂. In the presence of thrombin, the fibrin of peptides, A and B is cleaved, leading to the formation of soluble fibrin monomers (_{αβγ}) that form twisting fibrils and fibers of a three dimensional network. In the presence of factor XIII and calcium as catalysts, cross-linking occurs to form the insoluble form that is the final form of the fibriri sealant. Additional factors that can influence the formation of this material include pH, fibronectin, and temperature. The final fibrin sealant is subject to fibrinolytic degradation by both endogenous and exogenous plasmin. Antifibrinolytics such as aprotinin, transexaminic acid, and ε-aminocarproic acid can be added to the mixture to reduce the rate of fibrinolysis and creation of fibrin degradation products. Fibrin-based sealants are available commercially under the trade name of Tisseel (Baxter Healthcare).

According to this embodiment, the adhesives of the embodiment form a simple mechanical barrier upon polymerization, without an inflammatory reaction. After a period of time, the adhesive would be bioabsorbed and the tube would become patent and the method would have produced temporary contraception.

Any method could be used to deliver the adhesive to the fallopian tube. In one embodiment of the invention, this could be done laproscopically by occluding the opening of the tube to the egg. In this embodiment, a laproscope would be inserted through an incision in the patient's abdomen. A steer able, guide wire/catheter will be inserted through the operating channel of the laproscope and inserted into the distal opening of a first fallopian tube. ((F) as in FIG. 1) The desired adhesive would then be injected into the fallopian tube opening. The adhesive composition is allowed to cure in situ, causing occlusion of the tube. After withdrawal from the first fallopian tube, the guide wire/catheter would then be inserted into the distal opening of the second fallopian tube. The desired adhesive would then be injected into the opening of the second fallopian tube. The adhesive composition again would be allowed to cure in situ, causing occlusion of the tube. The guide wire/catheter would then be withdrawn, and the laproscope apparatus would be withdrawn from the abdomen.

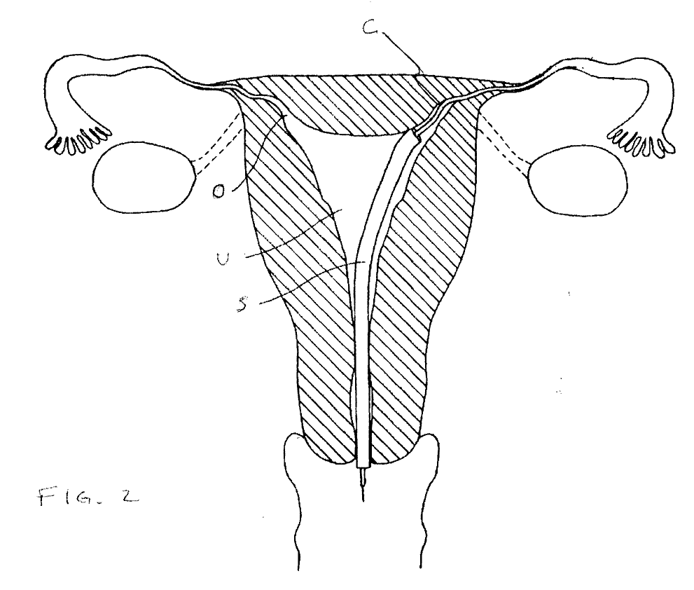

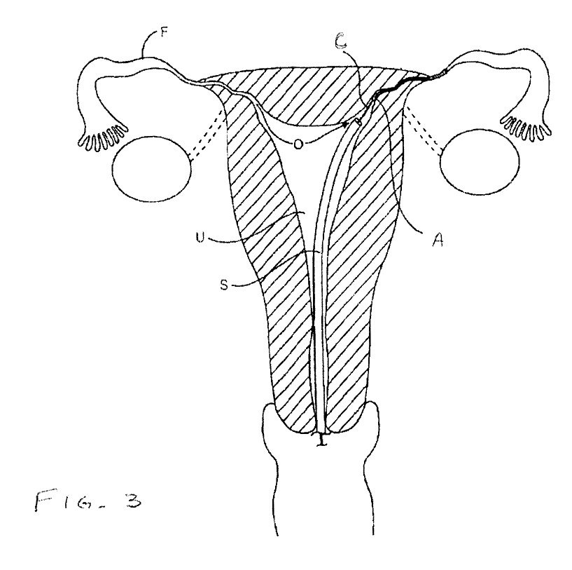

In another embodiment of the invention, the method could be performed conveniently in an office based, outpatient procedure. The first step in the procedure would involve placement of a hysteroscope transvaginally. Once in position, a camera is attached to the hysteroscope and the clinician has a clear view of the uterine cavity (U)(FIG. 2). Once the fallopian tubes ostia are visualized, or located, by the clinician, the catheter (C) is placed into the operating channel of the hysteroscope sheath (S) with the aid of a stiff steer able guide wire it is introduced into the ostia (O), as illustrated in FIG. 2. The stiffening wire is then removed from the catheter. Correct depth of insertion is indicated by preset markers on the catheter. A perfusion test can be performed to confirm catheter placement in the fallopian tube using 1 ml of methylene blue dye through an injection side port of the catheter. Once placement is confirmed the physician can then inject the desired adhesive into the first fallopian tube. The adhesive composition is allowed to cure in situ, causing the occlusion of the tube. Occlusion of the tube is illustrated in FIG. 3, wherein the adhesive (A) has occluded the fallopian tube. Using the same method the catheter could then be inserted into the ostium of the second fallopian tube, followed by injection of adhesive into the second fallopian tube. The adhesive composition again is allowed to cure in situ, causing occlusion of the second tube.

Any steer able directional catheter/guidewire could be used to penetrate the ostia and adhesive into the fallopian tube by either method described above, but the catheter may also have the following features. The catheter may have an open tip or sealed tip with perforations along the distal portion allowing even distribution in the tube. The catheter may have a curved distal portion to facilitate placement in the lumen and have preset or radiopaque markers. Most adhesives will be pre-mixed before injection but the catheter itself may have a mixing chamber. The catheter may also utilize a series of steer able stiffening wires or primary and secondary catheters to facilitate placement in the fallopian tube as describe above. The catheter may also utilize balloons or plugs to maintain the injected adhesive in the fallopian tube depending on the adhesive used and whether any detrimental effects would be expected from adhesive entering the peritoneal cavity.

In another embodiment of the invention, non-surgical permanent sterilization of females may be achieved by use of bioabsorbable tissue adhesives, which may include bioabsorbable cyanoacrylate adhesives, or a mixture of purified bovine serum albumin and glutaraldehyde, to provide occlusion of the fallopian tubes. Bioabsorbable cyanoacrylate adhesives are disclosed in U.S. Pat. No. 6,224,622 and pending U.S. application Ser. No. 11/124,831, the disclosures of which are herein incorporated by reference. In one such embodiment, biocompatible cyanoacrylate adhesives which provide occlusion of the fallopian tubes are alkyl 2-cyanoacrylate esters wherein the alkyl group is selected from the group consisting of C₂ to C₁₂ alkyl chains and the alkyl chain may be either a straight chain or a branched chain. In another such embodiment, the biocompatible cyanoacrylate adhesives are alkyl 2-cyanoacrylate esters wherein the alky group is selected from the group consisting of C₄ to C₁₂ alkyl chains and the alkyl chain may be either a straight chain or a branched chain.

Bioabsorbable adhesives which comprise mixtures of purified bovine serum albumin and glutaraldehyde are known by the name BioGlue, available from CryoLife Inc. Cross-linking and curing of the adhesive occurs with reaction of the amine groups of albumin with the aldehyde groups of glutaraldehyde. Reaction between the amine group present in tissue protein and the aldehyde groups of the adhesive provide covalent bonding of the adhesive to the tissue surface.

According to this embodiment, the adhesives of the embodiment would produce a mild inflammatory reaction in the fallopian tubes. Cells typically infiltrate the adhesive material as it is reabsorbed, laying down collagen as part of the normal healing process. The initial mechanical plug is then replaced with a functional scar plug leaving a permanent sterilization.

### DRAWINGS

### BRIEF DESCRIPTION OF THE DRAWINGS

For a more complete understanding of the features and objects of the invention, reference should be made to the following detailed description of the invention and the accompanying drawings, in which:

FIG. 1 illustrates the anatomy of the uterus and fallopian tubes for deployment of the contraceptive methods of the invention.

FIG. 2 illustrates a contraceptive method according to the invention.

FIG. 3 illustrates a fallopian tube and uterus after deployment of a contraceptive method of the invention

What is claimed is:

1. A method of female contraception comprising: injecting a bioabsorbable and biocompatible tissue adhesive into the fallopian tubes, and
allowing the said bioabsorbable tissue adhesive to polymerize, to occlude the said fallopian tubes (bilaterally) to achieve contraception.

2. A method as in claim 1 wherein the bioabsorbable tissue adhesive forms a simple mechanical barrier which would not produce a permanent scar tissue barrier after bioabsorbtion.

3. A method as in claim 1 wherein the bioabsorbable tissue adhesive produces a mild inflammatory reaction upon introduction which results in the formation of a permanent scar tissue plug upon bioabsorbtion of the said adhesive.

4. A method as in claim 2 wherein the bioabsorbable tissue adhesive is at least one adhesive selected from the group consisting of a mixture of polyethylene glycol macromers, and a fibrin-based sealant.

5. A method as in claim 2 wherein the bioabsorbable tissue adhesive is a mixture of polyethylene glycol macromers.

6. A method as in claim 2 wherein the bioabsorbable tissue adhesive is a fibrin-based sealant.

7. A method as in claim 3 wherein the bioabsorbable tissue adhesive is at least one adhesive selected from the group consisting of a bioabsorbable cyanoacrylate adhesive composition and a mixture of purified bovine serum albumin and glutaraldehyde.

8. A method as in claim 3 wherein the bioabsorbable tissue adhesive is a bioabsorbable alkyl 2-cyanoacrylate adhesive composition wherein the said alkyl group is selected from the group consisting of C₂ to C₁₂ alkyl chains.

9. A method as in claim 3 wherein the bioabsorbable tissue adhesive is a bioabsorbable alkyl 2-cyanoacrylate adhesive composition wherein the said alkyl group is selected from the group consisting of C₄ to C₁₂ alkyl chains.

10. A method as in claim 3 wherein the bioabsorbable tissue adhesive is a mixture of purified bovine serum albumin and glutaraldehyde.

11. A method as in claim 1 wherein the bioabsorbable tissue adhesive is injected into the fallopian tubes of a patient laproscopically to occlude the opening of each tube to an egg.

12. A method as in claim 1 wherein the bioabsorbable tissue adhesive is injected into the fallopian tubes using a hysteroscope.

13. A method of injecting a bioabsorbable adhesive into a fallopian tube comprising, inserting a hysteroscope into the uterus of a patient,
proceeding through the cervix of the said patient,
locating the uterine end of a first said fallopian tube,
visually inserting a catheter/guide wire from the hysteroscope into the ostium of the first said fallopian tube,
injecting the bioabsorbable adhesive into the first said fallopian tube,
allowing the said adhesive to cure in situ to form an occluding plug,
withdrawing said guide wire from the first fallopian tube,
locating the uterine end of a second said fallopian tube,
visually inserting a guide wire from the hysteroscope into the ostium of the second said fallopian tube,
injecting the bioabsorbable adhesive into the second said fallopian tube,
allowing the said adhesive to cure in situ to form an occluding plug,
withdrawing said guide wire from the first fallopian tube, and
withdrawing said hysteroscope.

14. A method as in claim 13 wherein the bioabsorbable adhesive forms a simple mechanical barrier which would not produce a permanent scar tissue barrier after bioabsorbtion.

15. A method as in claim 13 wherein the bioabsorbable tissue adhesive produces a mild inflammatory reaction upon introduction which results in the formation of a permanent scar tissue plug upon bioabsorbtion.

16. A method as in claim 14 wherein the bioabsorbable tissue adhesive is at least one adhesive selected from the group consisting of a mixture of polyethylene glycol macromers, and a fibrin-based sealant.

17. A method as in claim 14 wherein the bioabsorbable tissue adhesive is a mixture of polyethylene glycol macromers.

18. A method as in claim 14 wherein the bioabsorbable tissue adhesive is a fibrin-based sealant.

19. A method as in claim 15 wherein the bioabsorbable tissue adhesive is at least one adhesive selected from the group consisting of a bioabsorbable cyanoacrylate adhesive composition and a mixture of purified bovine serum albumin and glutaraldehyde.

20. A method as in claim 15 wherein the bioabsorbable tissue adhesive is a bioabsorbable cyanoacrylate adhesive composition.

21. A method as in claim 15 wherein the bioabsorbable tissue adhesive is a mixture of purified bovine serum albumin and glutaraldehyde.

22. A method of using a laproscope to inject a bioabsorbable tissue adhesive into the openings of the fallopian tubes of a patient comprising; making an incision in the abdomen of a patient,
inserting the said laproscope into the said abdomen,
locating and visually guiding said laproscope into the distal opening of the first said fallopian tube,
injecting a bioabsorbable and biocompatible tissue adhesive into the said distal opening of the fallopian tube,
permitting the adhesive to cure in situ to form an occluding plug,
withdrawing said laproscope from the first said fallopian tube,
locating and visually guiding said laproscope into the distal opening of the second said fallopian tube,
injecting a bioabsorbable and biocompatible tissue adhesive into the said distal opening of the fallopian tube,
permitting the adhesive to cure in situ to form an occluding plug,
withdrawing said laproscope from the second said fallopian tube, and
withdrawing said laproscope from said abdomen.

23. A method as in claim 22 wherein the bioabsorbable adhesive forms a simple mechanical barrier which would not produce a permanent scar tissue barrier after bioabsorbtion.

24. A method as in claim 22 wherein the bioabsorbable tissue adhesive produces a mild inflammatory reaction upon introduction which results in the formation of a permanent scar tissue plug upon bioabsorbtion.

25. A method as in claim 23 wherein the bioabsorbable tissue adhesive is at least one adhesive selected from the group consisting of a mixture of polyethylene glycol macromers, and a fibrin-based sealant.

26. A method as in claim 23 wherein the bioabsorbable tissue adhesive is a mixture of polyethylene glycol macromers.

27. A method as in claim 23 wherein the bioabsorbable tissue adhesive is a fibrin-based sealant.

28. A method as in claim 24 wherein the bioabsorbable tissue adhesive is at least one adhesive selected from the group consisting of a bioabsorbable cyanoacrylate adhesive composition and a mixture of purified bovine serum albumin and glutaraldehyde.

29. A method as in claim 24 wherein the bioabsorbable tissue adhesive is a bioabsorbable cyanoacrylate adhesive composition.

30. A method as in claim 24 wherein the bioabsorbable tissue adhesive is a mixture of purified bovine serum albumin and glutaraldehyde.

31. A kit for non-surgically occluding an oviduct with formed in place bioabsorbable adhesive plugs comprising in combination:
a syringe device comprising an adhesive composition, and
a directional guide wire allowing injection for insertion into a fallopian tube.

32. A kit as in claim 31 wherein, the syringe device is a dual syringe device.

33. A kit as in claim 32 wherein the adhesive composition is at least one adhesive selected from the group consisting of a mixture of polyethylene glycol macromers, a fibrin-based sealant, and a mixture of purified bovine serum albumin and glutaraldehyde.

34. A kit as in claim 33 wherein the adhesive composition is a mixture of polyethylene glycol macromers.

35. A kit as in claim 33 wherein the adhesive composition is a fibrin-based sealant.

36. A kit as in claim 33 wherein the adhesive composition is a mixture of purified bovine serum albumin and glutaraldehyde.

37. A kit as in claim 32 wherein the syringe device further comprises a mixing element.

38. A kit as in claim 31 wherein the syringe device is a single syringe device.

39. A kit as in claim 38 wherein the adhesive a bioabsorbable cyanoacrylate adhesive composition.

40. A kit as in claim 39 wherein the bioabsorbable cyanoacrylate tissue adhesive is a bioabsorbable alkyl 2-cyanoacrylate adhesive composition wherein the said alkyl group is selected from the group consisting of C₂ to C₁₂ alkyl chains.

41. A method as in claim 39 wherein the bioabsorbable tissue adhesive is a bioabsorbable alkyl 2-cyanoacrylate adhesive composition wherein the said alkyl group is selected from the group consisting of C₄ to C₁₂ alkyl chains.
